(19) United States
(12) Patent Application Publication
(10) Pub. No.: US 2022/0022514 A1
(43) Pub. Date: Jan. 27, 2022

(54) LIQUID CONCENTRATES FORMULATED FOR DILUTION INTO NUTRITIONAL PRODUCTS TO PROMOTE SAFE SWALLOWING FOR INDIVIDUALS WITH DYSPHAGIA

(71) Applicant: SOCIETE DES PRODUITS NESTLE S.A., Vevey (CH)

(72) Inventors: Adam Burbidge, Arzier (CH); Michael Jedwab, Lausanne (CH); Jan Engmann, Lausanne (CH)

(21) Appl. No.: 17/413,108

(22) PCT Filed: Dec. 3, 2019
(86) PCT No.: PCT/EP2019/083464

§ 371 Date: Jun. 11, 2021

Related U.S. Application Data
(60) Provisional application No. 62/779,087, filed on Dec. 13, 2018.

(51) Int. Cl. A23L 33/125; A23L 29/206; A23L 29/269; A23L 33/105

(52) U.S. Cl. CPC A23L 33/125; A23L 29/206; A23V 2002/00; A23L 33/105; A23L 29/271

(57) ABSTRACT

A liquid concentrate formulated for dilution into a nutritional product is disclosed, as well as the resultant nutritional product, uses thereof, methods for the manufacture thereof, and methods for improving the cohesiveness of the nutritional product. The nutritional products have improved cohesiveness for promoting safer swallowing of food boluses for individuals having swallowing difficulties such as dysphagia. In a preferred embodiment, the nutritional product is prepared by diluting a liquid concentrate of one or more of a beta-glucan, a plant-extracted gum such as okra gum, or a plant-derived mucilage. Preferably, the resultant nutritional product has a shear viscosity greater than 200 mPas and up to about 2,000 mPas, preferably greater than 200 mPas and up to about 500 mPas, more preferably 250 mPas to about 450 mPas, most preferably 250 mPas to about 400 mPas, when measured at a shear rate of 50 s⁻¹ at a temperature of 20° C.

### FIELD

The present disclosure is related to a liquid concentrate formulated for dilution into a nutritional product and also relates to the resultant nutritional product, a use of the nutritional product, a method for making the nutritional product and a method for improving the cohesiveness of the nutritional product. More specifically, the present disclosure relates to cohesive liquids in concentrated form.

### BACKGROUND

Dysphagia is a medical term for the symptom of difficulty in swallowing. Dysphagia may be a sensation that suggests a difficulty in a passage of solids or liquids (i.e. nutritional products) from the mouth to the stomach.

During processing in the mouth and swallowing, a viscosity of the nutritional products changes due to shear forces. It is known that in most of the cases the viscosity of nutritional products decreases when the shear forces and rate acting on the nutritional products (e.g., chewing forces) increase. Individuals who suffer from dysphagia often require thickened nutritional products. Thickening of the nutritional products is achieved to increase the viscosity of the food product by adding thickeners such as starch or gum thickeners. The thickened nutritional products make it less likely that an individual with dysphagia will aspirate during passage of the nutritional products from the mouth to the stomach. Individuals with dysphagia may find that nutritional products cause coughing, spluttering or even choking and therefore thickened nutritional products enables the individuals who suffer from dysphagia to swallow safely. Although the addition of thickeners is thought to improve a bolus control and timing of swallowing, it is disliked by individuals who suffer from dysphagia due to the extra swallowing effort required and as the thickener leaves residues with high levels of viscosity resulting in undesirable organoleptic properties. Furthermore thickened nutritional products lack the cohesiveness that saliva provides to food boluses.

Epidemiological studies estimate a prevalence rate of 16% to 22% among individuals who suffer from dysphagia that are over 50 years of age.

Dysphagia is classified into three major types: oropharyngeal dysphagia, esophageal dysphagia and functional dysphagia.

Oropharyngeal dysphagia is generally not treatable with medication. Oropharyngeal dysphagia affects individuals of all ages, but is more prevalent in older individuals. Worldwide, oropharyngeal dysphagia affects approximately 22 million people over the age of 50. Oropharyngeal dysphagia is often a consequence of an acute event such as a stroke, brain injury, or surgery for oral or throat cancer. In addition, radiotherapy and chemotherapy may weaken the muscles and degrade the nerves associated with the physiology and nervous innervation of the swallow reflex. Oropharyngeal dysphagia is also common for individuals with progressive neuromuscular diseases, such as Parkinson's disease, to experience increasing difficulty in swallowing initiation. Representative causes of oropharyngeal dysphagia include those associated neurological illnesses (brainstem tumors, head trauma, stroke, cerebral palsy, Guillain-Barre syndrome, Huntington's disease, multiple sclerosis, polio, post-polio syndrome, Tardive dyskinesia, metabolic encephalopathies, amyotrophic lateral sclerosis, Parkinson's disease, dementia), infectious illnesses (diphtheria, botulism, Lyme disease, syphilis, mucositis [herpetic, cytomegalovirus, candida, etc.]), autoimmune illnesses (lupus, scleroderma, Sjogren's syndrome), metabolic illnesses (amyloidosis, cushing's syndrome, thyrotoxicosis, Wilson's disease), myopathic illnesses (connective tissue disease, dermatomyositis, myasthenia gravis, myotonic dystrophy, oculopharyngeal dystrophy, polymyositis, sarcoidosis, paraneoplastic syndromes, inflammatory myopathy), iatrogenic illnesses (medication side effects [e.g., chemotherapy, neuroleptics, etc.], post surgical muscular or neurogenic, radiation therapy, corrosive [pill injury, intentional]), and structural illnesses (cricopharyngeal bar, Zenker's diverticulum, cervical webs, oropharyngeal tumors, osteophytes and skeletal abnormalities, congenital [cleft palate, diverticulae, pouches, etc.]).

Esophageal dysphagia can affect individuals of all ages. Esophageal dysphagia is generally treatable with medications and is considered a less serious form of dysphagia. Esophageal dysphagia is often a consequence of mucosal, mediastinal, or neuromuscular diseases. Mucosal (intrinsic) diseases narrow the lumen through inflammation, fibrosis, or neoplasia associated with various conditions (e.g., peptic stricture secondary to gastroesophageal reflux disease, esophageal rings and webs [e.g., sideropenic dysphagia or Plummer-Vinson syndrome], esophageal tumors, chemical injury [e.g., caustic ingestion, pill esophagitis, sclerotherapy for varices], radiation injury, infectious esophagitis, and eosinophilic esophagitis). Mediastinal (extrinsic) diseases obstruct the esophagus by direct invasion or through lymph node enlargement associated with various conditions (tumors [e.g., lung cancer, lymphoma], infections [e.g., tuberculosis, histoplasmosis], and cardiovascular [dilated auricula and vascular compression]). Neuromuscular diseases may affect the esophageal smooth muscle and its innervation, disrupting peristalsis or lower esophageal sphincter relaxation, or both, commonly associated with various conditions (achalasia [both idiopathic and associated with Chagas disease], scleroderma, other motility disorders, and a consequence of surgery [i.e., after fundoplication and antireflux interventions]). It is also common for individuals with intraluminal foreign bodies to experience acute esophageal dysphagia.

Functional dysphagia is defined in some patients wherein no organic cause for dysphagia can be found.

Dysphagia is not generally diagnosed. Dysphagia has major consequences on health and healthcare costs on individuals who suffer from dysphagia. Individuals who suffer from severe dysphagia experience a sensation of impaired passage of nutritional products from the mouth to the stomach, occurring immediately after swallowing. Among community dwelling individuals, perceived symptoms may bring the individuals who suffer from dysphagia to see a doctor. Among institutionalized individuals, health care practitioners may observe symptoms or hear comments from the individuals who suffer from dysphagia or their family member suggestive of swallowing impairment and recommend the individuals who suffer from dysphagia be evaluated by a specialist. As the general awareness of swallowing impairments is low among front-line practitioners, dysphagia often goes undiagnosed and untreated. Yet, through referral to a swallowing specialist (e.g., speech language pathologist), a patient can be clinically evaluated and dysphagia diagnosis can be determined.

The general awareness of swallowing impairments is low among front-line practitioners. Many people (especially those who are elderly) suffer with undiagnosed and untreated swallowing impairments. One reason is that front-line community care practitioners (e.g., general practitioners/geriatricians, home care nurses, physical therapists, etc.) do not typically screen for the condition. If they are aware of the severity of swallowing impairments, they commonly do not use an evidence-based method of screening.

A severity of dysphagia may vary from: (i) minimal (perceived) difficulty in safely swallowing nutritional products, (ii) an inability to swallow nutritional products without significant risk for aspiration or choking, and (iii) a complete inability to swallow nutritional products. An inability to properly swallow nutritional products may be due to food boluses of the nutritional products being broken up into smaller fragments, which may enter the airway or leave unwanted residues in the oropharyngeal and/or esophageal tract during the swallowing process (e.g., aspiration). If enough material enters the lungs, it is possible that the patient may drown on the nutritional products that have built up in the lungs. Even small volumes of aspirated nutritional products may lead to bronchopneumonia infection, and chronic aspiration may lead to bronchiectasis and may cause some cases of asthma.

Silent aspiration is a common condition among the elderly and refers to the aspiration of the oropharyngeal contents during sleep. People may compensate for less-severe swallowing impairments by self-limiting the diet. The aging process itself, coupled with chronic diseases such as hypertension or osteoarthritis, predisposes the elderly to (subclinical) dysphagia that may go undiagnosed and untreated until a clinical complication such as pneumonia, dehydration, malnutrition (and related complications) occurs.

Dysphagia and aspiration impacts upon quality of life, morbidity and mortality. Twelve-month mortality is high (45%) among individuals in institutional care who have dysphagia and aspiration. The economic burden of the clinical consequences arising from lack of diagnosis and early management of dysphagia are therefore significant.

As noted, pneumonia is a common clinical consequence of dysphagia. Pneumonia may require acute hospitalisation and emergency room visits. Among those that develop pneumonia due to aspiration, the differential diagnosis of ‘aspiration pneumonia’ is not necessarily indicated as a result of current care practices. Based on U.S. healthcare utilisation surveys from recent years, pneumonia accounted for over one million hospital discharges and an additional 392,000 were attributable to aspiration pneumonia. Individuals who have general pneumonia as the principal diagnosis have a mean 6 day hospital length of stay and incur over $18,000 in costs for hospital care. It is expected that aspiration pneumonia would carry higher costs for hospital care, based on a mean 8 day length of hospital stay. Pneumonia is life threatening among persons with dysphagia, the odds of death within 3 months is about 50% (van der Steen et al. 2002). In addition, an acute insult such as pneumonia often initiates the downward spiral in health among elderly. An insult is associated with poor intakes and inactivity, resulting in malnutrition, functional decline, and frailty. Specific interventions (e.g., to promote oral health, help restore normal swallow, or reinforce a swallow-safe bolus) would benefit persons at risk for (due to aspiration of oropharyngeal contents, including silent aspiration) or experiencing recurrent pneumonia.

Similar to pneumonia, dehydration is a life-threatening clinical complication of dysphagia. Dehydration is a common co-morbidity among hospitalised individuals with neurodegenerative diseases (thus, likely to have a swallowing impairment). The conditions of Alzheimer's disease, Parkinson's disease, and multiple sclerosis account for nearly 400,000 U.S. hospital discharges annually, and up to 15% of these patients suffer dehydration. Having dehydration as the principal diagnosis is associated with a mean 4 day length of hospital stay and over $11,000 in costs for hospital care. Nevertheless, dehydration is an avoidable clinical complication of dysphagia.

Malnutrition and related complications (e.g., [urinary tract] infections, pressure ulcers, increased severity of dysphagia [need for more-restricted food options, tube feeding, and/or PEG placement and reduced quality of life], dehydration, functional decline and related consequences [falls, dementia, frailty, loss of mobility, and loss of autonomy]) can arise when swallowing impairment leads to fear of choking on food and liquids, slowed rate of consumption, and self-limited food choices. If uncorrected, inadequate nutritional intake exacerbates dysphagia as the muscles that help facilitate normal swallow weaken as physiological reserves are depleted. Malnutrition is associated with having a more than 3-times greater risk of infection. Infections are common in individuals with neurodegenerative diseases (thus, likely to have a chronic swallowing impairment that jeopardizes dietary adequacy). The conditions of Alzheimer's disease, Parkinson's disease, and multiple sclerosis account for nearly 400,000 U.S. hospital discharges annually, and up to 32% of these patients suffer urinary tract infection.

Malnutrition has serious implications for patient recovery. Malnourished patients have longer length of hospital stay, are more likely to be re-hospitalised, and have higher costs for hospital care. Having malnutrition as the principal diagnosis is associated with a mean 8 day length of hospital stay and nearly $22,000 in costs for hospital care. Furthermore, malnutrition leads to unintentional weight loss and predominant loss of muscle and strength, ultimately impairing mobility and the ability to care for oneself With the loss of functionality, caregiver burden becomes generally more severe, necessitating informal caregivers, then formal caregivers, and then institutionalisation. However, malnutrition is an avoidable clinical complication of dysphagia.

Among persons with neurodegenerative conditions (e.g., Alzheimer's disease), unintentional weight loss (a marker of malnutrition) precedes cognitive decline. In addition, physical activity can help stabilize cognitive health. Thus, it is important to ensure nutritional adequacy among persons with neurodegenerative conditions to help them have the strength and endurance to participate in regular therapeutic exercise and guard against unintentional weight loss, muscle wasting, loss of physical and cognitive functionality, frailty, dementia, and progressive increase in caregiver burden.

Falls and related injuries are a special concern among elderly with neurodegenerative conditions, associated with loss of functionality. Falls are the leading cause of injury deaths among older adults. Furthermore, fall-related injuries among elderly accounted for more than 1.8M U.S. emergency room visits in a recent year. Direct medical costs totaled $179M for fatal and $19.3B for nonfatal fall-related injuries in the period of a year. As an effect of an ambitious non-payment for performance initiative introduced in U.S. hospitals in October 2008, Medicare will no longer pay hospitals for treatment cost of falls and related injuries that occur during the hospital stay. Hospitals will face a loss of about $50,000 for each elderly patient who falls and suffers hip fracture while in hospital care. This new quality initiative is based on the premise that falls are an avoidable medical error. In other words, falls are preventable within reason by applying evidence-based practices including medical nutrition therapy as nutritional interventions are efficacious in the prevention of falls and related injuries (e.g., fractures) among the elderly.

Chewing and swallowing difficulties are recognised risk factors for pressure ulcer development. Pressure ulcers are considered an avoidable medical error, preventable within reason by applying evidence-based practices (including nutritional care, as pressure ulcers are more likely when nutrition is inadequate). Pressure ulcers are a significant burden to the health care system. In U.S. hospitals in 2006, there were 322,946 cases of medical error connected with pressure ulcer development. The average cost of healing pressure ulcers depends on the stage, ranging from about $1,100 (for stage II) to about $10,000 (for stage III & IV pressure ulcers). Thus, the estimated cost of healing the cases of medical error connected with pressure ulcer development in one year, is in the range of $323M to $3.2B. As an effect of an ambitious non-payment for performance initiative introduced in U.S. hospitals in October 2008, Medicare will no longer pay hospitals for treatment cost of pressure ulcers that develop during the hospital stay (up to $3.2B annually). Pressure ulcers are preventable within reason, in part, by assuring nutritional intakes are adequate. Furthermore, specific interventions including the use of specialised nutritional supplements help reduce the expected time to heal pressure ulcers once they've developed.

In U.S. long-term care facilities, quality of care standards are enforced via the frequent regulatory survey. Surveyors will consider facilities out of compliance when they uncover evidence of actual or potential harm/negative outcomes. The range of penalties includes fines, forced closure, as well as lawsuits and settlement fees. The Tag F325 (nutrition) survey considers significant unplanned weight change, inadequate food/fluid intake, impairment of anticipated wound healing, failure to provide a therapeutic diet as ordered, functional decline, and fluid/electrolyte imbalance as evidence for providing sub-standard nutritional care. The Tag F314 (pressure ulcers) survey mandates that the facility must ensure that a resident who is admitted without pressure ulcers does not develop pressure ulcers unless deemed unavoidable. In addition, that a resident having pressure ulcers receives necessary treatment and services to promote healing, prevent infection and prevent new pressure ulcers from developing.

Therefore considering the prevalence of dysphagia and the possible complications related thereto, and the costs associated with same, it would be beneficial to provide nutritional products that promote safer swallowing of boluses of the nutritional products in individuals who suffer from dysphagia. Such nutritional products would improve the lives of a large and growing number of individuals who suffer from dysphagia. Specific interventions (e.g., to promote oral health, help restore normal swallowing, or reinforce a swallow-safe bolus) can enable individuals to eat orally as opposed to being tube fed and/or requiring PEG placement) and experience the psycho-social aspects of nutritional products associated with general well-being while guarding against the potentially negative consequences that result from lack of adequate swallowing ability. Improvements in the intake of nutritional products by individuals who suffer from dysphagia may also enable such individuals to swallow a wider variety of nutritional products safely and comfortably, which may lead to an overall healthier condition of the individual and prevent further health-related decline.

There is therefore a need to overcome the aforementioned drawbacks and to provide natural cohesiveness that saliva provides to food boluses of nutritional products when being consumed by an individual.

### SUMMARY

The present disclosure is related to a liquid concentrate formulated for dilution into a nutritional product and also relates to the resultant nutritional product, a use of the nutritional product, a method for making the nutritional product and a method for improving the cohesiveness of the nutritional product. More specifically, the present disclosure relates to cohesive liquids in concentrated form. To the best knowledge of the present inventors, the present disclosure provides cohesive liquids in concentrated form for the first time.

In a first aspect, the present disclosure provides a liquid concentrate comprising a beta-glucan and formulated for dilution into a nutritional product to thereby increase the shear viscosity of the nutritional product to a level that is greater than 200 mPas and up to about 2,000 mPas, preferably greater than 200 mPas and up to about 500 mPas, more preferably 250 mPas to about 450 mPas, most preferably 250 mPas to about 400 mPas, all values measured at a shear rate of 50 s⁻¹ at 20° C. Furthermore the amount of the beta-glucan provides to the nutritional product a relaxation time that can be determined by a Capillary Breakup Extensional Rheometry (CaBER) experiment of preferably more than 10 ms (milliseconds) at a temperature of typically about 20° C.

In another aspect, the present disclosure provides a nutritional product. The nutritional product is made by diluting a liquid concentrate which comprises a beta-glucan. The beta-glucan is present in the nutritional product in an amount that provides to the nutritional product a shear viscosity greater than 200 mPas and up to about 2,000 mPas, preferably greater than 200 mPas and up to about 500 mPas, more preferably 225 mPas to about 450 mPas, most preferably 250 mPas to about 400 mPas, all values measured at a shear rate of 50 s⁻¹ at 20° C. Furthermore the amount of the beta-glucan provides to the nutritional product a relaxation time that can be determined by a Capillary Breakup Extensional Rheometry (CaBER) experiment of preferably more than 10 ms (milliseconds) at a temperature of typically about 20° C.

In a further aspect, the nutritional product is used for treating a swallowing disorder in a patient in need of such treatment.

In a further aspect, the nutritional product is used for promoting safe swallowing of nutritional products in a patient in need of same.

In a further aspect, the nutritional product is used for mitigating the risks of aspiration during swallowing of nutritional products in a patient in need of same.

In another aspect, the present disclosure provides a method for making a nutritional product. The method comprises diluting a liquid concentrate comprising a beta-glucan. An amount of the liquid concentrate that is diluted provides to the nutritional product a shear viscosity greater than 200 mPas and up to about 2,000 mPas, preferably greater than 200 mPas and up to about 500 mPas, more preferably 225 mPas to about 450 mPas, most preferably 250 mPas to about 400 mPas, all values measured at a shear rate of 50 s⁻¹ at 20° C. Furthermore the amount of the liquid concentrate that is diluted provides to the nutritional product a relaxation time that can be determined by a Capillary Breakup Extensional Rheometry (CaBER) experiment of preferably more than 10 ms (milliseconds) at a temperature of typically about 20° C.

In a further aspect, the present disclosure provides a method for improving the cohesiveness of a nutritional product. The method comprises adding to a nutritional product a liquid concentrate comprising a beta-glucan. An amount of the liquid concentrate that is added to the nutritional product provides to the nutritional product a shear viscosity greater than 200 mPas and up to about 2,000 mPas, preferably greater than 200 mPas and up to about 500 mPas, more preferably 225 mPas to about 450 mPas, most preferably 250 mPas to about 400 mPas, all values measured at a shear rate of 50 s⁻¹ at 20° C. Furthermore the amount of the liquid concentrate that is added to the nutritional product provides to the nutritional product a relaxation time that can be determined by a Capillary Breakup Extensional Rheometry (CaBER) experiment of preferably more than 10 ms (milliseconds) at a temperature of typically about 20° C.

In another aspect, the present disclosure provides a system for production of a homogenous single phase beverage for administration to an individual having dysphagia, the system comprising a container comprising a liquid concentrate comprising a beta-glucan. The liquid concentrate is formulated for dilution into a nutritional product to thereby increase the shear viscosity of the nutritional product to a level that is greater than 200 mPas and up to about 2,000 mPas, preferably greater than 200 mPas and up to about 500 mPas, more preferably 225 mPas to about 450 mPas, most preferably 250 mPas to about 400 mPas, all values measured at a shear rate of 50 s⁻¹ at 20° C. Furthermore, the amount of the liquid concentrate that is diluted provides to the nutritional product a relaxation time that can be determined by a Capillary Breakup Extensional Rheometry (CaBER) experiment of preferably more than 10 ms (milliseconds) at a temperature of typically about 20° C.

In a further aspect, the system container comprises a metering pump connected to the container and configured to dispense an amount of the liquid concentrate per pump that is approximately equal to a predetermined amount; and preferably the number of pumps corresponds to an amount of the liquid concentrate suitable for a level of dysphagia in the individual. For example, one pump can dispense an amount of the liquid concentrate suitable for dilution into a nutritional product suitable for an individual with mild dysphagia, two pumps can dispense an amount of the liquid concentrate suitable for dilution into a nutritional product suitable for an individual with moderate dysphagia, and one pump can dispense an amount of the liquid concentrate suitable for dilution into a nutritional product suitable for an individual with severe dysphagia.

Additionally or alternatively to the beta-glucan, the liquid concentrate can comprise at least one plant-extracted gum selected from the group consisting of okra gum, konjac mannan, tara gum, locust bean gum, guar gum, fenugreek gum, tamarind gum, cassia gum, acacia gum, gum ghatti, pectins, cellulosics, tragacanth gum, karaya gum, and combinations thereof; and/or at least one plant-derived mucilage selected from the group consisting of cactus mucilage, psyllium mucilage, mallow mucilage, flax seed mucilage, marshmallow mucilage, ribwort mucilage, mullein mucilage, cetraria mucilage, and combinations thereof. In some embodiments, the liquid concentrate comprises a thickening component that is a beta-glucan together with one or two plant-extracted gums, and in some embodiments the thickening component is a beta-glucan together with one or two plant-derived mucilages.

The further embodiments describe the preferred aspects of the embodiments provided by the present disclosure.

### DETAILED DESCRIPTION

The various aspects and embodiments according to the present disclosure, as set forth herein, are illustrative of the specific ways to make and use the invention and do not limit the scope of invention when taken into consideration with the claims and the detailed description. It will also be appreciated that features from aspects and embodiments of the invention may be combined with further features from the same or different aspects and embodiments of the invention.

As used in this detailed description and the appended claims, the singular forms “a,” “an” and “the” include plural referents unless the context clearly dictates otherwise. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.” Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components. “Consisting essentially of” means that the embodiment or component thereof comprises more than 50 wt. % of the individually identified components, preferably at least 75 wt. % of the individually identified components, more preferably at least 85 wt. % of the individually identified components, most preferably at least 95 wt. % of the individually identified components, for example at least 99 wt. % of the individually identified components.

All ranges described are intended to include all numbers, whole or fractions, contained within the said range. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth. As used herein, wt. % refers to the weight of a particular component relative to total weight of the referenced composition.

In a first aspect, a liquid concentrate comprises a beta-glucan and is formulated for dilution in a diluent to form a nutritional product to thereby increase the shear viscosity of the nutritional product to a level that is greater than 200 mPas and up to about 2,000 mPas, preferably greater than 200 mPas and up to about 500 mPas, more preferably 225 mPas to about 450 mPas, most preferably 250 mPas to about 400 mPas, all values measured at a shear rate of 50 s⁻¹ at 20° C. In some embodiments, the shear viscosity is about 350 mPas or about 400 mPas at a shear rate of 50 s⁻¹ at 20° C. It is appreciated that measurement of shear viscosity can be performed at a different shear rate, for example another shear rate from 0 to 100 s⁻¹, or at different temperature, for example another temperature from 0 to 100° C.; nevertheless, such measurements must be related back to the standard conditions of 50 s⁻¹ at 20° C. disclosed herein. Furthermore, the amount of the liquid concentrate (e.g., the amount of beta-glucan) provides to the nutritional product a relaxation time that can be determined by a Capillary Breakup Extensional Rheometry (CaBER) experiment of preferably more than 10 ms (milliseconds) at a temperature of typically about 20° C. In some embodiments, the liquid concentrate consists essentially of the thickening agent (e.g., a beta-glucan) and water; for example, the liquid concentrate can consist of the thickening agent (e.g., a beta-glucan) and water.

As used herein, a “liquid concentrate” is a liquid that is formulated to be diluted before administration. Further in this regard, the liquid concentrates disclosed herein are only administered after addition of another ingredient, such as a liquid diluent, preferably water. Moreover, the term “nutritional product” refers to a nutritional composition for oral administration by an individual who suffers from dysphagia. The nutritional product is envisaged for supplemental nutrition, for hydration, or for replacement of one or more full meals of the individual who suffers from dysphagia. The nutritional product is also understood to include any number of optional ingredients (e.g., ingredients additional to the liquid concentrate from which the nutritional product is made). Non-limiting examples of suitable optional ingredients include conventional food additives, for example one or more, acidulants, additional thickeners, buffers or agents for pH adjustment, chelating agents, colorants, emulsifiers, excipient, flavour agent, minerals, osmotic agents, a pharmaceutically acceptable carrier, preservatives, stabilisers, sugar(s), sweetener(s), texturiser(s), and/or vitamin(s). The optional ingredients can be added in any suitable amount. Preferably, the liquid concentrate is a homogeneous single phase liquid comprising water, and preferably the nutritional product is a homogeneous single phase beverage comprising water. Nevertheless, the present disclosure is not limited to a specific embodiment of the nutritional product. Furthermore, the present disclosure is not limited to a specific embodiment of the diluent in which the liquid concentrate is reconstituted, and the diluent can be any liquid suitable for consumption by an animal or human.

A “ready to drink” beverage or “RTD” beverage is a beverage in liquid form that can be consumed without further addition of liquid. Preferably an RTD beverage is aseptic. An “oral nutrition supplement” or “ONS” is a composition comprising at least one macronutrient and/or at least one micro nutrient, for example in a form of sterile liquids, semi-solids or powders, and intended to supplement other nutritional intake such as that from food. Non-limiting examples of commercially available ONS products include MERITENE®, BOOST®, NUTREN® and SUSTAGEN®.

The term “unit dosage form,” as used herein, refers to physically discrete units suitable as unitary dosages for human and animal subjects, each unit containing a predetermined quantity of the composition disclosed herein in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of liquid concentrate dispensed by a dispenser or housed within a container such as a pouch.

The term individual refers to any human, animal, mammal or who suffers from dysphagia that can benefit from the nutritional product. It is to be appreciated that animal includes, but is not limited to, mammals. Mammal includes, but is not limited to, rodents, aquatic mammals, domestic animals such as dogs and cats, farm animals such as sheep, pigs, cows and horses, and humans.

As used herein, a beta-glucan (β-glucan) refers to homopolysaccharides of D-glucopyranose monomers linked by (1→3), (1→4)-β-glycosidic bonds. A beta-glucan is derivable from plant or microbial origin, e.g. from oat or barley, by methods known to the skilled person, for example as described by Lazaridou et al. in ‘A comparative study on structure-function relations of mixed-linkage (1→3), (1→4) linear β-D-glucans’ in Food Hydrocolloids, 18 (2004), 837-855. Beta-glucan and hence also oat shows particularly preferable properties in the inventive compositions as it allows to provide with small amounts of beta-glucan the claimed higher shear viscosities greater than 200 mPas and up to about 2,000 mPas, preferably greater than 200 mPas and up to about 500 mPas, more preferably 225 mPas to about 450 mPas, most preferably 250 mPas to about 400 mPas, all values when measured at a shear rate of 50 s⁻¹ and at a temperature of 20° C.; and relaxation times of more than 10 milliseconds, preferably as determined by a CABER experiment at a temperature of 20° C. as described herein.

Additionally or alternatively to the beta-glucan, the liquid concentrate can comprise a plant-extracted gum selected from the group consisting of okra gum, konjac mannan, tara gum, locust bean gum, guar gum, fenugreek gum, tamarind gum, cassia gum, acacia gum, gum ghatti, pectins, cellulosics, tragacanth gum, karaya gum, and combinations thereof and/or a plant-derived mucilages selected from the group consisting of cactus mucilage, psyllium mucilage, mallow mucilage, flax seed mucilage, marshmallow mucilage, ribwort mucilage, mullein mucilage, cetraria mucilage, and combinations thereof.

As used herein, the feature “bolus” includes any entity of the nutritional product formed in the mouth in preparation for swallowing. The bolus may be of any shape, size, composition and/or texture, and thus it may also be a liquid.

Shear viscosity of a nutritional product can be determined by any method that can accurately control the shear rate applied to the product and simultaneously determine the shear stress or vice versa. Standard methods include the use of concentric cylinders, cone-and-plate and plate-plate geometries. Relaxation times can be determined in this context by a Capillary Breakup Extensional Rheometry (CaBER) as further outlined below.

Shear viscosity is a measurable rheological property. Shear viscosity, often referred to as viscosity, describes the action of a material to applied shear stress. In other words, shear stress is the ratio between “stress” (force per unit area) exerted on the surface of a fluid, in the lateral or horizontal direction, to the change in velocity of the fluid as you move down in the fluid (a “velocity gradient”).

Another rheological property of a material is its extensional viscosity. Extensional viscosity is the ratio of the stress required to extend a liquid in its flow direction to the extension rate. Extensional viscosity coefficients are widely used for characterising polymers, where they cannot be simply calculated or estimated from the shear viscosity. Rheological studies are generally performed using rheometers, which generally impose a specific stress field or deformation to the fluid and monitor the resultant deformation or stress. These instruments may operate in steady flow or oscillatory flow, as well as both shear and extension.

The Capillary Breakup Extensional Rheometer (CaBER) is an example for a rheometer applying extensional stress. During the CaBER experiment as performed herein for measuring the relaxation time of the nutritional product, a drop of said product is placed between two vertically aligned and parallel circular metal surfaces, both having a diameter of 6 mm. The metal surfaces are then rapidly separated linearly over a time interval of 50 ms (milliseconds). The filament formed by this stretching action subsequently thins under the action of interfacial tension and the thinning process is followed quantitatively using a digital camera and/or laser sheet measuring the filament diameter at its mid-point. The relaxation time in a CaBER experiment is determined by plotting the normalised natural logarithm of the filament diameter during the thinning process versus time and determining the slope of the linear portion (dₗₙ(D/D₀)/dₜ) of this curve, where D is the filament diameter, D₀ the filament diameter at time zero and t the time of filament thinning. The relaxation time in this context is then defined as minus one third (−⅓) times the inverse of this slope, i.e. −1/(3dₗₙ(D/D₀)/dₜ).

It is preferred that in the present nutritional product, the relaxation time is less than 2000 ms, preferably from 20 ms to 1000 ms, likewise preferably from 50 ms to 500 ms, and more preferably from 50 ms to 200 ms, each at a temperature of 20° C. Even more preferably the relaxation time is between 75 ms to 200 ms at a temperature of 20° C. or most preferably even between 75 ms to 150 ms at a temperature of 20° C.

Moreover, in a preferred embodiment, a filament diameter of the nutritional product decreases less than linearly, and preferably exponentially in time during the CaBER experiment. The filament diameter can be measured using a digital camera and/or laser sheet measuring device.

In some embodiments, the liquid concentrate comprises a beta-glucan in a concentration from at least 0.01 wt. % to 25 wt. %, for example from at least 0.01 wt. % to 10 wt. %, preferably from at least 0.1 wt. % to 7.5 wt. %, and most preferably from at least 1 wt. % to 5 wt. %, likewise preferably e.g. from at least 0.1 wt. % to 10 wt. %, from at least 1 wt. % to 10 wt. %, from at least 5 wt. % to 10 wt. % or from at least 2.5 wt. % to 7.5 wt. %. Nevertheless, in some embodiments, the beta-glucan is greater than 25 wt. % of the liquid concentrate.

In a further embodiment the nutritional product is in an aqueous diluted form. In this regard, the nutritional product can be an aqueous dilution of the liquid concentrate disclosed herein in a ratio up to 200:1, preferably 50:1 to 100:1. It is to be understood that a dilution of 200:1 means 200 parts of diluent:1 part of liquid concentrate.

In some embodiments, the liquid concentrate further comprises water (in addition to the thickening component), and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

In some embodiments, the liquid concentrate is in a unit dosage form comprising an amount of the liquid concentrate effective for administration of the nutritional product to an individual who suffers from dysphagia to achieve at least one of (i) supplemental nutrition, (ii) hydration and (ii) replacement of one or more full meals.

The nutritional product may furthermore comprise one or more of a protein, a fat, a fiber, a carbohydrate, a prebiotic, a probiotic, an amino acid, a fatty acid, a phytonutrient, an antioxidant, and/or combinations thereof.

The protein can be a dairy-based protein, a plant-based protein or an animal-based protein or any combination thereof Dairy-based proteins include, for example, casein, caseinates (e.g., all forms including sodium, calcium, potassium caseinates), casein hydrolysates, whey (e.g., all forms including concentrate, isolate, demineralized), whey hydrolysates, milk protein concentrate, and milk protein isolate. Plant-based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, green pea powder, green bean powder, and any proteins derived from beans, lentils, and pulses. Animal-based proteins may be selected from the group consisting of beef, poultry, fish, lamb, seafood, or combinations thereof.

The fat can be vegetable fat (such as olive oil, corn oil, sunflower oil, rapeseed oil, hazelnut oil, soy oil, palm oil, coconut oil, canola oil, lecithins, and the like), animal fat (such as milk fat) or any combinations thereof.

The fiber can be a fiber blend that may contain a mixture of soluble and insoluble fiber. Soluble fibers may include, for example, fructooligosaccharides, acacia gum, inulin, and the like. Insoluble fibers may include, for example, pea outer fiber.

The carbohydrate can comprise sucrose, lactose, glucose, fructose, corn syrup solids, maltodextrin, modified starch, amylose starch, tapioca starch, corn starch or any combinations thereof.

The nutritional product can comprise at least one the following prebiotics or any combination thereof: acacia gum, alpha glucan, arabinogalactans, dextrans, fructooligosaccharides, fucosyllactose, galactooligosaccharides, galactomannans, gentiooligosaccharides, glucooligosaccharides, guar gum, inulin, isomalto-oligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof. The prebiotic is a food substance that selectively promotes the growth of beneficial bacteria or inhibits the growth or mucosal adhesion of pathogenic bacteria in the intestines. The prebiotic are not inactivated in the stomach and/or upper intestine or absorbed in the gastrointestinal tract of the individual ingesting them, but they are fermented by the gastrointestinal microflora and/or by probiotics. Prebiotics are, for example, defined by Glenn R. Gibson and Marcel B. Roberfroid, Dietary Modulation of the Human Colonic Microbiota: Introducing the Concept of Prebiotics, J. Nutr. 1995 125: 1401-1412.

The nutritional product can comprise at least one probiotic. Probiotics are food-grade microorganisms (alive, including semi-viable or weakened, and/or non-replicating), metabolites, microbial cell preparations or components of microbial cells that could confer health benefits on a host when administered, more specifically probiotics beneficially affect the host by improving intestinal microbial balance, leading to effects on the health or well-being of the host. See, Salminen S, Ouwehand A. Benno Y. et al., Probiotics: how should they be defined?, Trends Food Sci. Technol. 1999:10, 107-10. In general, it is believed that these probiotics inhibit or influence the growth and/or metabolism of pathogenic bacteria in the intestinal tract. The probiotics may also activate the immune function of the host. The probiotics may include Aerococcus, Aspergillus, Bacillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or any combination thereof.

The nutritional product may comprise a synbiotic. A synbiotic is a supplement that comprises both a prebiotic (at least one of the aforementioned) and a probiotic (at least one of the aforementioned) that work together to improve the microflora of the intestine.

The nutritional product can comprise at least one the following amino acids or any combination thereof: alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine and valine.

In a further embodiment, the nutritional product can comprise at least one fatty acid or any combination thereof, for example ω-3 fatty acids such α-linolenic acid (“ALA”), docosahexaenoic acid (“DHA”) and eicosapentaenoic acid (“EPA”). The fatty acid can be derived from fish oil, krill, poultry, eggs, a plant source, algae and/or a nut source, e.g., flax seed, walnuts, almonds.

The nutritional product can comprise at least one phytonutrient. The phytonutrient can be at least one of flavanoids, allied phenolic compounds, polyphenolic compounds, terpenoids, alkaloids, or sulphur-containing compounds. Phytonutrients are non-nutritive compounds that are found in many foods. Phytonutrients are functional foods that have health benefits beyond basic nutrition, and are health promoting compounds that come from plant sources. Phytonutrient refers to any chemical produced by a plant that imparts one or more health benefit on a user. Non-limiting examples of suitable phytonutrients include:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3 -gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins.

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta-sitosterol, gamma-sitosterol, stigmasterol), tocopherols (vitamin E), and γ-3, γ-6, and γ-9 fatty acids (such as, for example, gamma-linolenic acid); triterpenoid (such as, for example, oleanolic acid, ursolic acid, betulinic acid, moronic acid).

iii) betalains which include Betacyanins (such as: betanin, isobetanin, probetanin, neobetanin); and betaxanthins (non glycosidic versions) (such as, for example, indicaxanthin, and vulgaxanthin).

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide.

v) protein inhibitors, which include, for example, protease inhibitors.

vi) other organic acids which include oxalic acid, phytic acid (inositol hexaphosphate); tartaric acid; and anacardic acid.

The nutritional product can comprise at least one antioxidant. Antioxidants are molecules capable of slowing or preventing the oxidation of other molecules. The antioxidant can be any one of astaxanthin, carotenoids, coenzyme Q10 (“CoQ10”), flavonoids, glutathione Goji (wolfberry), hesperidin, lactowolfberry, lignan, lutein, lycopene, polyphenols, selenium, vitamin A, vitamin C, vitamin E, zeaxanthin, or any combinations thereof.

The nutritional product is preferably in an administrable form, for example an orally administrable form. The administrable form can be any one of a pharmaceutical formulation, a nutritional formulation, a dietary supplement, a functional food and a beverage product, or any combinations thereof.

The optional ingredients such as the mineral(s) includes boron, calcium, chromium, copper, iodine, iron, magnesium, manganese, molybdenum, nickel, phosphorus, potassium, selenium, silicon, tin, vanadium, zinc, or any combinations thereof.

The optional ingredients such as vitamin(s) includes vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, folic acid and biotin) essential in amounts for normal growth and activity of the body, or any combinations thereof.

In a further aspect, the nutritional product is used for treating a swallowing disorder in a patient in need of such treatment. As used herein, the terms treating, treatment, treat and alleviate include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms treating, treatment, treat and alleviate also refer to the maintenance and/or promotion of health in an individual not suffering from a disease but who may be susceptible to the development of an unhealthy condition, such as nitrogen imbalance or muscle loss. The terms treating, treatment, treat and alleviate are also intended to include the potentiation or otherwise enhancement of one or more primary prophylactic or therapeutic measure. The terms treating, treatment, treat and alleviate are further intended to include the dietary management of a disease or condition or the dietary management for prophylaxis or prevention a disease or condition.

In a further aspect, the nutritional product is used for promoting safe swallowing of nutritional products in a patient in need of same.

In a further aspect, the nutritional product is used for mitigating the risks of aspiration during swallowing of nutritional products in a patient in need of same.

In a further aspect, a method for making the nutritional product comprises providing a liquid concentrate comprising a beta-glucan in a concentration from 0.01 wt. % to 25 wt. % capable of providing to the nutritional product: a shear viscosity greater than 200 mPas and up to about 2,000 mPas (e.g., greater than 200 mPas and up to about 500 mPas, or 250 mPas to about 400 mPas) when measured at a shear rate of 50 s⁻¹ at a temperature of 20° C., and a relaxation time, determined by a Capillary Breakup Extensional Rheometry (CaBER) experiment, of more than 10 ms (milliseconds) at a temperature of 20° C.

In a further aspect, a method for improving the cohesiveness of a nutritional product comprises adding to a nutritional product a liquid concentrate comprising a beta-glucan in a concentration from 0.01 wt. % to 25 wt. % capable of providing to the nutritional product: a shear viscosity up to 2,000 mPas (e.g., greater than 200 mPas and up to about 500 mPas, or 250 mPas to about 400 mPas) when measured at a shear rate of 50 s⁻¹ at a temperature of 20° C., and a relaxation time, determined by a Capillary Breakup Extensional Rheometry (CaBER) experiment, of more than 10 ms (milliseconds) at a temperature of 20° C.

In some embodiments of these methods, the nutritional product has a shear viscosity greater than 200 mPas and up to about 2,000 mPas, preferably greater than 200 mPas and up to about 500 mPas, more preferably 225 mPas to about 450 mPas, most preferably 250 mPas to about 400 mPas, all values when measured at a shear rate of 50 s⁻¹ at a temperature of 20° C. In some embodiments of these methods, the nutritional product has a shear viscosity of about 350 mPas or about 400 mPas, when measured at a shear rate of 50 s⁻¹.

In some embodiments of these methods, the relaxation time is less than 2000 ms, preferably from 20 ms to 1000 ms, likewise preferably from 50 ms to 500 ms, and more preferably from 50 ms to 200 ms, each at a temperature of 20° C. Even more preferably the relaxation time is between 75 ms to 200 ms at a temperature of 20° C. or most preferably even between 75 ms to 150 ms at a temperature of 20° C.

In some embodiments of these methods, a filament diameter of the nutritional product decreases less than linearly, and preferably exponentially in time during the CaBER experiment.

In some embodiments of these methods, the liquid concentrate comprises a beta-glucan in a concentration from at least 0.01 wt. % to 25 wt. %, preferably from at least 0.1 wt. % to 15 wt. %, and most preferably from at least 1 wt. % to 10 wt. %.

In some embodiments of these methods, the nutritional product is in an aqueous diluted form. In this regard, the nutritional product can be an aqueous dilution of the liquid concentrate disclosed herein ranging from 2:1 to 50:1, more preferably from 3:1 to 20:1 and most preferably from 5:1 to 10:1. It is to be understood that a dilution of 2:1 means 2 parts of diluent:1 part of liquid concentrate.

In some embodiments of these methods, the method further comprises adding one or more of a protein, a fat, a fiber, a carbohydrate, a prebiotic, a probiotic, an amino acid, a fatty acid, a phytonutrient, an antioxidant, and/or combinations thereof into the nutritional product.

In some embodiments of these methods, at least one protein is added to the nutritional product. The at least one protein can be a dairy-based protein, a plant-based protein or an animal-based protein or any combination thereof. Dairy-based proteins include, for example, casein, caseinates (e.g., all forms including sodium, calcium, potassium caseinates), casein hydrolysates, whey (e.g., all forms including concentrate, isolate, demineralized), whey hydrolysates, milk protein concentrate, and milk protein isolate. Plant-based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, green pea powder, green bean powder, and any proteins derived from beans, lentils, and pulses. Animal-based proteins may be selected from the group consisting of beef, poultry, fish, lamb, seafood, or combinations thereof.

In a preferred embodiment, the liquid concentrate that is diluted into the nutritional product can be easily dispersed by fork or eating utensil or any other simple method of agitation such as shaking by hand or by machine. In a preferred embodiment, the liquid concentrate consistently and rapidly thickens most fluids that a patient with dysphagia would desire or have a need to consume. This process provides a food thickener that is more palatable and more visually appealing and that is safer to have in the presence of patients with impaired decision making.

In a preferred embodiment, the liquid concentrate is prepared from water and a thickener selected from the group consisting of a beta-glucan, a plant-extracted gum such as okra gum, a plant-derived mucilage, and combinations thereof. Water of any quality may be employed; however, an exemplary embodiment includes tap potable water.

The amount of thickener (e.g., a beta-glucan, a plant-extracted gum, and/or a plant-derived mucilage) employed in a concentrate thickener will depend greatly on the specific thickener chosen, its specific thickening properties, and the processing equipment employed. In general, the amount employed can be between about 1% and about 25% thickener by weight, for example between about 1% and about 10% thickener by weight.

Typically a sufficient quantity of thickener powder for the concentrate being prepared is admixed with diluent in a suitable mixing vessel. A preferred mixing vessel can comprise a container having a size accommodating the amounts of thickener powder and diluent desired to be suitably mixed. The vessel can be a commercially sized tank which may optionally include a cover, a particular shape, baffles, and/or a heat jacket. Other suitable useful mixing vessels include a drinking cup, bowls, household containers which can be opened or closed, a kitchen top mixer system, as well as any suitably sized container which can accommodate the amount of diluent and thickener to be suitably admixed.

Generally, the temperature of the diluent is not critical to the preparation of the concentrate thickener and may include, without limitation: hot, cold, or room temperature diluent. With some particular thickeners, the inherent properties will make the choice of temperature more critical than with others.

As necessary or desired, minor components such as acids, bases, acidulates, chelating agents, flavors, colors, vitamins, minerals, sweeteners, insoluble foods and/or preservatives may be incorporated into the thickener and diluent admixture at any appropriate point during the preparation. Such minor components are preferably present in minor amounts and concentrations, i.e. a non-substantial amount as relates to thickening.

In an exemplary embodiment, depending on the specific admixing equipment used and the appropriate handling of the materials, the time for admixing the thickener concentrate is from about 2 minutes to about 180 minutes and preferably from about 5 minutes to about 60 minutes, although greater and lesser times may be employed if desired or necessary.

Optionally, as necessary or desired, the thickener concentrate may be treated to provide shelf stability. Most commonly, but not limiting, the treatment is heat in combination with one or more of the minor components mentioned above.

The packaging of the thickener concentrate is not critical as long as it delivers an effective amount of concentrate to thicken a liquid food to a thickness effective for a person afflicted with dysphagia. Illustratively, packaging may be totes, bins, foil pouches, buckets, bags, syringes or the like. If desired, use of a thickener concentrate can facilitate in-line mixing and preparation of thickened beverages in a beverage dispenser or container. Such a system can include a metering device and an in-line mixing system to dispense thickened beverages. Preferably the system is designed to dispense thickened or non-thickened beverages at the turn of a switch.

In an aspect, the thickener concentrate is an effective thickener for liquid foods. For example, an effective amount of the thickener concentrate can admixed with a liquid food which illustratively is selected from at least one of milk, human breast milk, cow's milk, soda, coffee, tea, juice (lemon, citrus, orange, apple), alcohol (beer, wine, or mixed drinks with less than about 20% alcohol), nutritional supplements, mixtures thereof and the like or a soup, broth, or food puree and the like. As used herein, the term “juice” includes puree, fruit juices including orange juice, vegetable juice and apple juice strained and unstrained, concentrated and fresh.

Non-limiting examples of suitable vessels to effectively admix the thickener concentrate and the liquid food include drinking cups, coffee cups, bowls, household containers which can be open top or closed top, a kitchen blender, a kitchen top mixer system, as well as any suitably sized container which can accommodate the materials to be admixed. Non-limiting examples of suitable instruments to carry out the admixing include forks, spoons, knives, hand mixers, kitchen blenders, kitchen top mixers, whisks, and any other appropriate agitation devices. Particularly suitable mixing containers have a lid or cover that can be attached to the container to allow the liquid food and the thickener concentrate to be shaken together with containment.

In an exemplary process, the amount of thickener concentrate employed in the admixture is that amount which provides a thickened liquid food which is capable of being consumed by effectively swallowing by a person afflicted with dysphagia.

In an exemplary process, the amount of time employed in the admixing disclosed herein is in the range from about 0.01 to about 3 minutes and preferably from about 0.01 minutes to about 1.5 minutes. Advantageously, the amount of mixing time is preferably such that staffing in hospitals, skilled nursing facilities, nursing homes, and/or retirement homes are able to more comfortably accommodate. Also advantageously, the thickened liquid food is preferably fully thickened once the concentrate is fully diluted. In such embodiments, no standing time is necessary because the polymer dissolution is completed during the preparation of the concentrate. These advantages are improvements over the current commercial products which require a two step process during the preparation of the final foodstuff which combines mixing and standing time. These products typically require at least about 30 minutes to fully and properly thicken the liquid food.

Another advantage is that the nutritional products resulting from the thickener concentrates disclosed herein are safer to eat without adding any further liquid to them and they are safer to leave in the presence of persons with impaired mental judgment. Although certainly very viscous, consumption of the thickener concentrate without dilution does not present a choking hazard. Dry powders put in the mouth and/or attempted to be swallowed before dissolving could present a danger to a patient with impaired mental judgment. In many facilities, open containers of powder are left on tables or in rooms or individual sized packets are served on trays. If a caregiver is somehow distracted, an impulsive eater, such as an individual afflicted with Huntington's chorea, could quickly try to consume the dry powder, at serious risk. The nutritional products disclosed herein are completely hydrated and thus face no such problems.

Another advantage of this approach to thickening liquid foods is the consistency of preparation. In contrast, dry powders for thickening at home or institutions typically have variability of the preparations.

The thickener concentrates disclosed herein can be delivered to the end user fully, completely, and totally hydrated, and may minimize or avoid settling or separation when shipped. Preferably, the density will not change over time, and the product is stable. Consequently, in such embodiments, the same volume of thickener concentrate would thicken a liquid food to the same level of thickness whether the thickener concentrate is from the top or the bottom of a container. Liquid foods thickened by a thickener concentrate preferably do not continue to thicken after preparation. The thickener can be already hydrated in the thickener concentrate, and thus any concern over the fluid environment and its impact on hydration time is minimized or eliminated.

A radiological technique known commonly as the modified barium swallow can be used to diagnose and to make therapeutic recommendations on thickened diets to those patients afflicted with dysphagia. Currently, hospitals or nursing homes or mobile diagnostic units prepare the test solutions in their own manner. There is little standardization on the thickness of these solutions. There are no means in place to ensure that the mealtime preparations served to diagnosed patients actually are the same thickness as the test preparations.

The thickener concentrate compositions disclosed herein can provide the opportunity to link the thicknesses prepared during the modified barium swallow to what is prepared in food service and/or bedside and/or at home. The thickener concentrate compositions disclosed herein can reduce the variability of final thickness in different liquid foods and thus reduce the variability of mixing technique. The elimination of clumping and mixing time factors can reduce the variability between what happens during a modified barium swallow and in food service and/or bedside and/or at home for actual consumption.

Another common diagnostic technique of dysphagia is the fiberoptic endoscopic evaluation of swallow. In this technique, an endoscope is inserted through the patient's nasal passage into the throat to directly observe the patient's swallow function. In an aspect, thickening concentrates disclosed herein can be used to thicken test preparations used in this evaluation technique.

In some embodiments of the methods disclosed herein, the method comprises identifying a level of severity of the swallowing disorder in the patient; and selecting, based on the level of severity of the swallowing disorder in the patient, the amount of the liquid concentrate for diluting, wherein the amount of the liquid concentrate is selected from a plurality of predetermined amounts that each corresponds to a different level of swallowing disorder severity. As a non-limiting example, the liquid concentrate can be provided in a container attached to a metering pump; one pump of the metering pump can dispense a predetermined amount of the liquid concentrate that is suitable for an individual with mild dysphagia, two pumps of the metering pump can dispense a predetermined amount of the liquid concentrate that is suitable for an individual with moderate dysphagia, and three pumps of the metering pump can dispense a predetermined amount of the liquid concentrate that is suitable for an individual with severe dysphagia.

In another aspect, the present disclosure provides a use of a liquid concentrate and another liquid in preparation of an orally administrable nutritional product consisting essentially of the liquid concentrate and the other liquid, the other liquid is suitable for consumption by an individual without dysphagia, the orally administrable nutritional product is suitable for administration to an individual having dysphagia, the liquid concentrate comprising a thickening component selected from the group consisting of a beta-glucan, a plant-extracted gum, a plant-derived mucilage and combinations thereof in a concentration from 0.01 wt. % to 25 wt. %, the liquid concentrate provides to the nutritional product: a shear viscosity greater than 200 mPas and up to about 500 mPas at a shear rate of 50 s⁻¹ at 20° C. (e.g., greater than 200 mPas and up to about 2,000 mPas, preferably greater than 200 mPas and up to about 500 mPas, more preferably 225 mPas to about 450 mPas, most preferably 250 mPas to about 400 mPas), and a relaxation time, determined by a Capillary Breakup Extensional Rheometry (CaBER) experiment, of more than 10 ms (milliseconds) at a temperature of 20° C.

In another aspect, the present disclosure provides a system for production of a homogenous single phase beverage for administration to an individual having dysphagia, the system comprising: a container comprising a liquid concentrate comprising a beta-glucan in a concentration from 0.01 wt. % to 25 wt. %, the liquid concentrate formulated for dilution into a nutritional product to thereby increase the shear viscosity of the nutritional product to a level between 200 and about 500 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C.; and a metering pump connected to the container and configured to dispense an amount of the liquid concentrate per pump performed on the metering pump by a user that is approximately equal to a predetermined amount. In an embodiment, a number of pumps performed on the metering pump by a user corresponds to an amount of the liquid concentrate suitable for a level of dysphagia in the individual. The metering pump can be configured to dispense an amount of the liquid concentrate that has a volume of 1-200 ml, preferably 1-100 ml, most preferably 1-50 ml. The system can further comprise a static in-line mixer configured to mix the liquid concentrate into the nutritional product and/or a nozzle configured to dispense the homogenous single phase beverage.

In yet another aspect, the present disclosure provides a liquid concentrate comprising a thickening component selected from the group consisting of a beta-glucan, at least one plant-extracted gum, at least one plant-derived mucilage, and combinations thereof in a concentration greater than 10 wt. % and up to 25 wt. %. The liquid concentrate is formulated for dilution into a nutritional product to provide to the nutritional product: a shear viscosity below 200 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C. In some embodiments, the concentration of the thickening component is greater than 12.5 wt. %, preferably greater than 15.0 wt. %. In some embodiments, the shear viscosity of the nutritional product is 10 mPas to 200 mPas, preferably 25 to 200 mPas, more preferably a shear viscosity from 50 to 200 mPas, even more preferably a shear viscosity from 75 to 200 mPas, yet more preferably from 100 to 200 mPas, or from 125 to 200 mPas, or from 150 to 200 mPas, or from 110 to 160 mPas, at a shear rate of 50 s⁻¹ at 20° C. In some embodiments, the liquid concentrate is diluted in diluent in a ratio greater than 50:1 and up to 200:1, for example 75:1 to 100:1.

Further aspects and embodiments of the invention are set out in the following lettered paragraphs:

A. A liquid concentrate comprising a thickening component selected from the group consisting of a beta-glucan, at least one plant-extracted gum, at least one plant-derived mucilage, and combinations thereof, the liquid concentrate is formulated for dilution in a diluent to form a nutritional product having:

a shear viscosity greater than 200 mPas and up to about 2,000 mPas at a shear rate of 50 s⁻¹ at 20° C., and

a relaxation time, determined by a Capillary Breakup Extensional Rheometry (CaBER) experiment, of more than 10 ms (milliseconds) at a temperature of 20° C.

B. The liquid concentrate of paragraph A, wherein the shear viscosity of the nutritional product is greater than 200 mPas and up to about 500 mPas when measured at a shear rate of 50 s⁻¹.

C. The liquid concentrate of paragraph A, wherein the shear viscosity of the nutritional product is from 250 mPas to about 400 mPas when measured at a shear rate of 50 s⁻¹.

D. The liquid concentrate of paragraph A, wherein the thickening component comprises a beta-glucan, preferably oat beta-glucan.

E. The liquid concentrate of paragraph A, wherein the liquid concentrate is diluted in the diluent in a ratio up to 200:1.

F. The liquid concentrate of paragraph A, wherein the liquid concentrate is diluted in the diluent in a ratio from 50:1 to 100:1

G. The liquid concentrate product of paragraph A, wherein the nutritional product is in an administrable form selected from the group consisting of pharmaceutical formulations, nutritional formulations, dietary supplements, functional food and beverage products, and combinations thereof.

H. The liquid concentrate of paragraph A, wherein the thickening component is 0.01 wt. % to 25 wt. % of the liquid concentrate.

I. The liquid concentrate of paragraph A, wherein the liquid concentrate further comprises water, and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

J. The liquid concentrate of paragraph A, wherein the liquid concentrate is diluted from a unit dosage form of the liquid concentrate that is an amount effective for administration of the nutritional product to an individual who suffers from dysphagia to achieve at least one of (i) supplemental nutrition, (ii) hydration or (ii) replacement of one or more full meals.

K. A nutritional product made by diluting in a diluent an amount of a liquid concentrate comprising a thickening component selected from the group consisting of a beta-glucan, at least one plant-extracted gum, at least one plant-derived mucilage, and combinations thereof, the amount of the thickening component in the nutritional product provides to the nutritional product:

a shear viscosity greater than 200 mPas and up to about 2,000 mPas at a shear rate of 50 s⁻¹ at 20° C., and

a relaxation time, determined by a Capillary Breakup Extensional Rheometry (CaBER) experiment, of more than 10 ms (milliseconds) at a temperature of 20° C.

L. The nutritional product of paragraph K, wherein the shear viscosity is greater than 200 mPas and up to about 500 mPas when measured at a shear rate of 50 s⁻¹.

M. The nutritional product of paragraph K, wherein the shear viscosity is from 250 mPas to about 400 mPas when measured at a shear rate of 50 s⁻¹.

N. The nutritional product of paragraph K, wherein the thickening component comprises a beta-glucan.

O. The nutritional product of paragraph K, in which the liquid concentrate is diluted in diluent in a ratio up to 200:1, preferably from 50:1 to 100:1.

P. The nutritional product of paragraph K further comprising one or more of a protein, a fat, a fiber, a carbohydrate, a prebiotic, a probiotic, an amino acid, a fatty acid, a phytonutrient, an antioxidant, and/or combinations thereof.

Q. The nutritional product of paragraph K, wherein the nutritional product is in an administrable form selected from the group consisting of pharmaceutical formulations, nutritional formulations, dietary supplements, functional food and beverage products, and combinations thereof.

R. The nutritional product of paragraph K, wherein the thickening component is 0.01 wt. % to 25 wt. % of the liquid concentrate.

S. The nutritional product of paragraph K, wherein the liquid concentrate further comprises water, and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

T. The nutritional product of paragraph K, wherein the liquid concentrate is diluted from a unit dosage form of the liquid concentrate that is an amount effective for administration of the nutritional product to an individual who suffers from dysphagia to achieve at least one of (i) supplemental nutrition, (ii) hydration or (ii) replacement of one or more full meals.

U. A method of treating a swallowing disorder in a patient in need thereof, the method comprising:

diluting an amount of a liquid concentrate into a nutritional product, the liquid concentrate comprising a thickening component selected from the group consisting of a beta-glucan, at least one plant-extracted gum, at least one plant-derived mucilage, and combinations thereof, the amount of the liquid concentrate that is diluted provides to the nutritional product a shear viscosity greater than 200 mPas and up to about 2,000 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C.; and

orally administering the nutritional product to the patient.

V. The method of paragraph U, wherein the shear viscosity is greater than 200 mPas and up to about 500 mPas at a shear rate of 50 s⁻¹ at 20° C.

W. The method of paragraph U, wherein the shear viscosity is from 250 mPas about 400 mPas at a shear rate of 50 s⁻¹ at 20° C.

X. The method of paragraph U, wherein the thickening component comprises a beta-glucan.

Y. The method of paragraph U, comprising:

identifying a level of severity of the swallowing disorder in the patient; and

selecting, based on the level of severity of the swallowing disorder in the patient, the amount of the liquid concentrate for diluting, wherein the amount of the liquid concentrate is selected from a plurality of predetermined amounts that each corresponds to a different level of swallowing disorder severity.

Z. The method of paragraph U, wherein the thickening component is 0.01 wt. % to 25 wt. % of the liquid concentrate.

A′. The method of paragraph U, wherein the liquid concentrate further comprises water, and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

B′. The method of paragraph U, wherein the liquid concentrate is diluted from a unit dosage form of the liquid concentrate that is an amount effective for administration of the nutritional product to an individual who suffers from dysphagia to achieve at least one of (i) supplemental nutrition, (ii) hydration or (ii) replacement of one or more full meals.

C′. A method of promoting safe swallowing of a nutritional product in a patient in need thereof, the method comprising:

diluting an amount of a liquid concentrate into the nutritional product, the liquid concentrate comprising a thickening component selected from the group consisting of a beta-glucan, at least one plant-extracted gum, at least one plant-derived mucilage, and combinations thereof, the amount of the liquid concentrate that is diluted provides to the nutritional product a shear viscosity greater than 200 mPas and up to about 2,000 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C.; and

orally administering the nutritional product to the patient.

D′. The method of paragraph C′, wherein the shear viscosity is greater than 200 mPas and up to about 500 mPas when measured at a shear rate of 50 s⁻¹.

E′. The method of paragraph C′, wherein the shear viscosity is from 250 to about 400 mPas when measured at a shear rate of 50 s⁻¹.

F′. The method of paragraph C′, wherein the thickening component comprises a beta-glucan.

G′. The method of paragraph C′, comprising:

identifying a level of severity of the swallowing disorder in the patient; and

selecting, based on the level of severity of the swallowing disorder in the patient, the amount of the liquid concentrate for diluting, wherein the amount of the liquid concentrate is selected from a plurality of predetermined amounts that each corresponds to a different level of swallowing disorder severity.

H′. The method of paragraph C′, wherein the thickening component is 0.01 wt. % to 25 wt. % of the liquid concentrate.

I′. The method of paragraph C′, wherein the liquid concentrate further comprises water, and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

J′. The method of paragraph C′, wherein the liquid concentrate is diluted from a unit dosage form of the liquid concentrate that is an amount effective for administration of the nutritional product to an individual who suffers from dysphagia to achieve at least one of (i) supplemental nutrition, (ii) hydration or (ii) replacement of one or more full meals.

K′. A method of mitigating a risk of aspiration during swallowing of a nutritional product in a patient in need thereof, the method comprising:

diluting an amount of a liquid concentrate into the nutritional product, the liquid concentrate comprising a thickening component selected from the group consisting of a beta-glucan, at least one plant-extracted gum, at least one plant-derived mucilage, and combinations thereof, the amount of the liquid concentrate that is diluted provides to the nutritional product a shear viscosity greater than 200 mPas and up to about 2,000 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C.; and

orally administering the nutritional product to the patient.

L′. The method of paragraph K′, wherein the shear viscosity is greater than 200 mPas and up to about 500 mPas at a shear rate of 50 s⁻¹ at 20° C.

M′. The method of paragraph K′, wherein the shear viscosity is from 250 to about 400 mPas at a shear rate of 50 s⁻¹ at 20° C.

N′. The method of paragraph K′, wherein the thickening component comprises a beta-glucan.

O′. The method of paragraph K′, comprising:

identifying a level of severity of the swallowing disorder in the patient; and

selecting, based on the level of severity of the swallowing disorder in the patient, the amount of the liquid concentrate for diluting, wherein the amount of the liquid concentrate is selected from a plurality of predetermined amounts that each corresponds to a different level of swallowing disorder severity.

P′. The method of paragraph K′, wherein the thickening component is 0.01 wt. % to 25 wt. % of the liquid concentrate.

Q′. The method of paragraph K′, wherein the liquid concentrate further comprises water, and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

R′. The method of paragraph K′, wherein the liquid concentrate is diluted from a unit dosage form of the liquid concentrate that is an amount effective for administration of the nutritional product to an individual who suffers from dysphagia to achieve at least one of (i) supplemental nutrition, (ii) hydration or (ii) replacement of one or more full meals.

S′. A method of making a nutritional product, the method comprising diluting an amount of a liquid concentrate into the nutritional product, the liquid concentrate comprising a thickening component selected from the group consisting of a beta-glucan, at least one plant-extracted gum, at least one plant-derived mucilage, and combinations thereof, the amount of the liquid concentrate that is diluted provides to the nutritional product a shear viscosity greater than 200 mPas and up to about 2,000 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C.

T′. The method of paragraph S′, wherein the shear viscosity is greater than 200 mPas and up to about 500 mPas at a shear rate of 50 s⁻¹ at 20° C.

U′. The method of paragraph S′, wherein the shear viscosity is from 250 to about 400 mPas at a shear rate of 50 s⁻¹ at 20° C.

V′. The method of paragraph S′, wherein the thickening component comprises a beta-glucan.

W′. The method of paragraph S′, comprising:

identifying a level of severity of the swallowing disorder in the patient; and

selecting, based on the level of severity of the swallowing disorder in the patient, the amount of the liquid concentrate for diluting, wherein the amount of the liquid concentrate is selected from a plurality of predetermined amounts that each corresponds to a different level of swallowing disorder severity.

X′. The method of paragraph S′, wherein the thickening component is 0.01 wt. % to 25 wt. % of the liquid concentrate.

Y′. The method of paragraph S′, wherein the liquid concentrate further comprises water, and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

Z′. The method of paragraph S′, wherein the liquid concentrate is diluted from a unit dosage form of the liquid concentrate that is an amount effective for administration of the nutritional product to an individual who suffers from dysphagia to achieve at least one of (i) supplemental nutrition, (ii) hydration or (ii) replacement of one or more full meals.

A″. A method of improving cohesiveness of a nutritional product, the method comprising diluting an amount of a liquid concentrate into the nutritional product, the liquid concentrate comprising a thickening component selected from the group consisting of a beta-glucan, at least one plant-extracted gum, at least one plant-derived mucilage, and combinations thereof, the amount of the liquid concentrate that is diluted provides to the nutritional product a shear viscosity greater than 200 mPas and up to about 2,000 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C.

B″. The method of paragraph A″, wherein the shear viscosity is greater than 200 mPas and up to about 500 mPas at a shear rate of 50 s⁻¹ at 20° C.

C″. The method of paragraph A″, wherein the shear viscosity is from 250 to about 400 mPas at a shear rate of 50 s⁻¹ at 20° C.

D″. The method of paragraph A″, wherein the thickening component comprises a beta-glucan.

E″. The method of paragraph A″, comprising: identifying a level of severity of the swallowing disorder in the patient; and

selecting, based on the level of severity of the swallowing disorder in the patient, the amount of the liquid concentrate for diluting, wherein the amount of the liquid concentrate is selected from a plurality of predetermined amounts that each corresponds to a different level of swallowing disorder severity.

F″. The method of paragraph A″, wherein the thickening component is 0.01 wt. % to 25 wt. % of the liquid concentrate.

G″. Use of a liquid concentrate and another liquid in preparation of an orally administrable nutritional product consisting essentially of the liquid concentrate and the other liquid, the other liquid is suitable for consumption by an individual without dysphagia, the orally administrable nutritional product is suitable for administration to an individual having dysphagia, the liquid concentrate comprising a thickening component selected from the group consisting of a beta-glucan, at least one plant-extracted gum, at least one plant-derived mucilage, and combinations thereof, the liquid concentrate provides to the nutritional product:

a shear viscosity greater than 200 mPas and up to about 2,000 mPas at a shear rate of 50 s⁻¹ at 20° C., and

a relaxation time, determined by a Capillary Breakup Extensional Rheometry (CaBER) experiment, of more than 10 ms (milliseconds) at a temperature of 20° C.

H″. A system for production of a homogenous single phase beverage for administration to an individual having dysphagia, the system comprising:

a container comprising a liquid concentrate comprising a thickening component selected from the group consisting of a beta-glucan, at least one plant-extracted gum, at least one plant-derived mucilage, and combinations thereof, the liquid concentrate formulated for dilution into a nutritional product to thereby increase the shear viscosity of the nutritional product to a level greater than 200 mPas and up to about 2,000 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C.; and

a metering pump connected to the container and configured to dispense an amount of the liquid concentrate per pump performed on the metering pump by a user that is approximately equal to a predetermined amount.

I″. The system of paragraph H″, wherein a number of pumps performed on the metering pump by a user corresponds to an amount of the liquid concentrate suitable for a level of dysphagia in the individual.

J″. The system of paragraph H″, further comprising a static in-line mixer configured to mix the liquid concentrate into the nutritional product.

K″. The system of paragraph H″, further comprising a nozzle configured to dispense the homogenous single phase beverage.

L″. The system of paragraph H″, wherein the metering pump is configured to dispense an amount of the liquid concentrate that has a volume of 1-200 ml, preferably 1-100 ml, most preferably 1-50 ml.

M″. A liquid concentrate comprising a thickening component selected from the group consisting of a beta-glucan, at least one plant-extracted gum, at least one plant-derived mucilage, and combinations thereof in a concentration greater than 10 wt. % and up to 25 wt. %, the liquid concentrate is formulated for dilution into a nutritional product to provide to the nutritional product:

a shear viscosity below 200 mPas at a shear rate of 50 s⁻¹ at 20° C., and

a relaxation time, determined by a Capillary Breakup Extensional Rheometry (CaBER) experiment, of more than 10 ms (milliseconds) at a temperature of 20° C.

N″. The liquid concentrate of paragraph M″, wherein the concentration of the thickening component is greater than 12.5 wt. %, preferably greater than 15.0 wt. %.

O″. The liquid concentrate of paragraph M″, wherein the shear viscosity of the nutritional product is 10 mPas to 200 mPas, preferably 25 to 200 mPas, more preferably a shear viscosity from 50 to 200 mPas, even more preferably a shear viscosity from 75 to 200 mPas, yet more preferably from 100 to 200 mPas, or from 125 to 200 mPas, or from 150 to 200 mPas, or from 110 to 160 mPas, at a shear rate of 50 s⁻¹ at 20° C.

P″. The liquid concentrate of paragraph M″, wherein the liquid concentrate is diluted in diluent in a ratio greater than 50:1 and up to 200:1, preferably from 75:1 to 100:1.

Q″. The liquid concentrate of paragraph M″, wherein the liquid concentrate further comprises water, and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

R″. The liquid concentrate of paragraph M″, wherein the liquid concentrate is diluted from a unit dosage form of the liquid concentrate that is an amount effective for administration of the nutritional product to an individual who suffers from dysphagia to achieve at least one of (i) supplemental nutrition, (ii) hydration or (ii) replacement of one or more full meals.

Having thus described the present invention and the advantages thereof, it should be appreciated that the various aspects and embodiments of the present invention as disclosed herein are merely illustrative of specific ways to make and use the invention. The various aspects and embodiments of the present invention do not limit the scope of the invention when taken into consideration with the appended claims and the forgoing detailed description.

1. A liquid concentrate comprising a thickening component selected from the group consisting of a beta-glucan, at least one plant-extracted gum, at least one plant-derived mucilage, and combinations thereof, the liquid concentrate is formulated for dilution in a diluent to form a nutritional product having:
a shear viscosity greater than 200 mPas and up to about 2,000 mPas at a shear rate of 50 s⁻¹ at 20° C., and
a relaxation time, determined by a Capillary Breakup Extensional Rheometry (CaBER) experiment, of more than 10 ms (milliseconds) at a temperature of 20° C.

2. The liquid concentrate according to claim 1, wherein the shear viscosity of the nutritional product is greater than 200 mPas and up to about 500 mPas when measured at a shear rate of 50 s⁻¹.

3. (canceled)

4. The liquid concentrate according to claim 1, wherein the thickening component comprises a beta-glucan.

5. The liquid concentrate according to claim 1, wherein the liquid concentrate is diluted in the diluent in a ratio up to 200:1.

6. The liquid concentrate product according to claim 1, wherein the nutritional product is in an administrable form selected from the group consisting of pharmaceutical formulations, nutritional formulations, dietary supplements, functional food and beverage products, and combinations thereof.

7. (canceled)

8. The liquid concentrate of according to claim 1, wherein the liquid concentrate further comprises water, and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

9-10. (canceled)

11. A method of treating a swallowing disorder or mitigating a risk of aspiration during swallowing of a nutritional product, in a patient in need thereof, the method comprising:
diluting an amount of a liquid concentrate into a nutritional product, the liquid concentrate comprising a thickening component selected from the group consisting of a beta-glucan, at least one plant-extracted gum, at least one plant-derived mucilage, and combinations thereof, the amount of the liquid concentrate that is diluted provides to the nutritional product a shear viscosity greater than 200 mPas and up to about 2,000 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C.; and
orally administering the nutritional product to the patient.

12. (canceled)

13. The method according to claim 11, wherein the shear viscosity is greater than 200 mPas and up to about 500 mPas at a shear rate of 50 s⁻¹ at 20° C.

14. The method according to claim 11, wherein the thickening component comprises a beta-glucan.

15. The method according to claim 11, comprising:
identifying a level of severity of the swallowing disorder in the patient; and
selecting, based on the level of severity of the swallowing disorder in the patient, the amount of the liquid concentrate for diluting, wherein the amount of the liquid concentrate is selected from a plurality of predetermined amounts that each corresponds to a different level of swallowing disorder severity.

16. The method according to claim 11, wherein the thickening component is 0.01 wt. % to 25 wt. % of the liquid concentrate.

17. The method according to claim 11, wherein the liquid concentrate further comprises water, and the nutritional product has at least one formulation selected from the group consisting of (i) the diluent is one or more of water, milk, a beverage comprising water and further comprising at least one component additional to the water, a liquid oral nutritional supplement (ONS), or a food product, and the dilution of the liquid concentrate in the diluent directly forms the nutritional product such that the nutritional product consists essentially of or consists of the diluent, the thickening component, and the water from the liquid concentrate; (ii) the dilution of the liquid concentrate in the diluent forms an aqueous solution followed by addition of the aqueous solution to at least one other orally administrable composition to form the nutritional product, such that the nutritional product consists essentially of or consists of the diluent, the thickening component, the water from the liquid concentrate, and the at least one other orally administrable composition; and (iii) the nutritional product is a ready-to-drink beverage made by packaging the nutritional product after the dilution of the liquid concentrate in the diluent.

18. The method according to claim 11, wherein the liquid concentrate is diluted from a unit dosage form of the liquid concentrate that is an amount effective for administration of the nutritional product to an individual who suffers from dysphagia to achieve at least one of (i) supplemental nutrition, (ii) hydration or (ii) replacement of one or more full meals.

19-20. (canceled)

21. A system for production of a homogenous single phase beverage for administration to an individual having dysphagia, the system comprising:
a container comprising a liquid concentrate comprising a thickening component selected from the group consisting of a beta-glucan, at least one plant-extracted gum, at least one plant-derived mucilage, and combinations thereof, the liquid concentrate formulated for dilution into a nutritional product to thereby increase the shear viscosity of the nutritional product to a level greater than 200 mPas and up to about 2,000 mPas at a shear rate of 50 s⁻¹ at 20° C., and a relaxation time, determined by a Capillary Breakup Extensional Rheometry (CaBER) experiment, of more than 10 ms (milliseconds) at a temperature of 20° C.; and
a metering pump connected to the container and configured to dispense an amount of the liquid concentrate per pump performed on the metering pump by a user that is approximately equal to a predetermined amount.

22. The system according to claim 21, wherein a number of pumps performed on the metering pump by a user corresponds to an amount of the liquid concentrate suitable for a level of dysphagia in the individual.

23. The system according to claim 21, further comprising a static in-line mixer configured to mix the liquid concentrate into the nutritional product.

24. The system according to claim 21, further comprising a nozzle configured to dispense the homogenous single phase beverage.

25-31. (canceled)
